(19) United States
(12) Patent Application Publication
(10) Pub. No.: US 2013/0150455 A1
(43) Pub. Date: Jun. 13, 2013

(54) METHOD FOR TREATING MECHANICAL ALLODYNIA COMPRISING ADMINISTRATION OF EUGENOL

(71) Applicants: Seog Bae OH, Seoul (KR); Sung Jun JUNG, Suwon-si (KR); Ge Hoon CHUNG, Seoul (KR); Yong Ho KIM, Seoul (KR)

(72) Inventors: Seog Bae OH, Seoul (KR); Sung Jun JUNG, Suwon-si (KR); Ge Hoon CHUNG, Seoul (KR); Yong Ho KIM, Seoul (KR)

(73) Assignee: SNU R&DB FOUNDATION, Seoul (KR)

(21) Appl. No.: 13/494,859

(22) Filed: Jun. 12, 2012

(30) Foreign Application Priority Data
Dec. 7, 2011 (KR) 10-2011-0130590

(51) Int. Cl. A61K 31/085

(52) U.S. Cl. CPC A61K 31/085
USPC 514/720

(57) ABSTRACT

The present invention relates to a pharmaceutical composition for selectively treating mechanical allodynia, which provides eugenol or a pharmaceutically acceptable salt thereof at a concentration lower than that which would inhibit voltage-gated sodium channels (VGSCs), a pharmaceutical composition for blocking hyperpolarization-activated current (Iₕ), and a transdermal preparation for treating mechanical allodynia comprising the composition.

Even though administered at a concentration lower than that which would inhibit VGSCs, the eugenol of the present invention inhibits Iₕ in a cAMP or G-protein coupled receptor (GPCR) independent manner, thereby selectively ameliorating mechanical allodynia. Therefore, when the eugenol of the present invention is formulated into a transdermal preparation to be provided at a concentration lower than that which would inhibit VGSCs, and directly applied to the wound lesion, it can be used as a pharmaceutical composition capable of selectively treating mechanical allodynia.

### CROSS-REFERENCE TO RELATED APPLICATION

This application claims priority to Korean Patent Application No. 10-2011-0130590, filed Dec. 7, 2011. This application is incorporated herein by reference in its entirety.

### BACKGROUND OF THE INVENTION

1. Field of the Invention

The present invention relates to a method for treating mechanical allodynia comprising administering to a patient a therapeutically effective amount of eugenol or a pharmaceutically acceptable salt thereof, wherein the concentration of the administered eugenol or a pharmaceutically acceptable salt thereof is lower than that which would inhibit voltage-gated sodium channels (VGSCs), a method for blocking hyperpolarization-activated current (Iₕ), and a transdermal preparation for treating mechanical allodynia.

2. Description of the Related Art

Pain is a useful phenomenon that protects life from internal and external risks. Pain can be largely divided into two categories; physiological pain and pathological pain. Physiological pain is a very important physiological function for survival, whereas pathological pain occurs after tissue damage or nerve injury, and is not a simple repetition of physiological pain. Pain caused by tissue damage or nerve injury is commonly called neuropathic pain, and is accompanied by long-term and chronic changes, including considerable changes in pain sensation. Although there is no reliable statistical data in Korea, the prevalence of chronic pain in France is more than 31%. Of those suffering from chronic pain, 20% have the characteristics of neuropathic pain, i.e. some 6% of the total population [Lanteri-Miet, et al., Presented at 11^{th} World Congress on Pain, Aug. 21-26, 2005, Sidney, Australia]. When this abnormal pain gets worse, quality of life becomes poorer, in some individuals leading to suicidal thoughts. Depending on the causes, neuropathic pain is classified into post-traumatic or postsurgical pain; metabolic pain such as diabetic neuropathy; ischaemic or hemorrhagic pain such as central pain after stroke; toxic pain such as heavy metal poisoning or chemotherapy; compression pain such as spinal stenosis or carpal tunnel syndrome; immune-mediated pain such as multiple sclerosis; inflammatory pain such as post-herpetic neuralgia; and hereditary pain such as Fabry's disease.

Pain can also be classified according to symptoms. Firstly, according to the presence of stimulation, it can be classified into stimulation-independent spontaneous pain and stimulation-dependent evoked pain. Of these, spontaneous pain can be divided into continuous and paroxysmal pain according to the type of pain generation, and divided into pain caused by non-noxious stimulus below the pain threshold and pain caused by noxious stimulus according to the stimulus intensity of evoked pain. Furthermore, pain caused by non-noxious stimulus can be divided into mechanical allodynia and thermal allodynia. According to the feature of the stimulus, mechanical allodynia is subdivided into dynamic allodynia and static allodynia, and thermal allodynia is subdivided into cold allodynia and heat allodynia. Meanwhile, pain caused by noxious stimulus can be divided into mechanical hyperalgesia and thermal hyperalgesia. According to the feature of the stimulus, mechanical hyperalgesia is subdivided into punctate hyperalgesia and pressure hyperalgesia, and thermal hyperalgesia is subdivided into cold hyperalgesia and heat hyperalgesia [Campbell, J. N. and Meyer, R. A., Neuron, 2006, 52: 77-92].

Although it is believed that peripheral and central mechanisms are involved in the pathogenesis of neuropathic pain, the mechanisms underlying the pathogenesis of spontaneous pain and mechanical allodynia remain incompletely understood [Biel, M. et al., Physiol. Rev., 2009, 89: 847-885; Jiang, Y. Q. et al., Neurochem. Res., 2008, 33: 1979-1989; Wickenden, A. D. et al., Curr. Pharm. Des., 2009, 15: 149-168]. Spontaneous ectopic discharges of injured primary sensory neurons and hyperexcitability of medium- or large-diameter myelinated fiber (Aβ) are thought to contribute to mechanical allodynia [Wickenden, A. D. et al., Curr. Pharm. Des., 2009, 15: 149-168]. Previous work has shown that altered expression levels of several types of voltage-gated sodium channels (VGSCs) contribute to the generation and persistence of spontaneous discharges [Matzner, O. and Devor, M., J. Neurophysiol., 1994, 72: 349-359; Waxman, S. G., Nat. Rev. Neurosci., 2001, 2: 652-659; Waxman, S. G. et al., Proc. Natl. Acad. Sci. USA, 1999, 96: 7635-7639]. Several findings suggest that hyperpolarization-activated cyclic nucleotide-gated (HCN) channels may also play an important role in neuropathic pain [Donlop, J. et al., Curr. Pharm. Des., 2009, 15: 1767-1772; Lee, D. H. et al., J. Pain, 2005, 6: 417-424].

In mammals, the HCN channel family comprises 4 members (HCN1-4) that are expressed in the heart and nervous system, and the current produced by HCN channels has been known to be I_{f} or Iₕ. Although hyperpolarization-activated current (Iₕ) was first designated as a pacemaker current in cardiac cells, Iₕ has been also identified in dorsal root ganglion (DRG) neurons, particularly in medium- and large-size neurons, where most ectopic discharges occur after peripheral nerve injury [Lee, D. H. et al., J. Pain, 2005, 6: 417-424; Momin, A. et al., J. Physiol., 2008, 586: 5911-5929]. In situ hybridization and immunohistochemistry revealed the presence of HCN channels in DRGs [Moosmang, S. et al., Eur. J. Biochem., 2001, 268: 1646-1652; Tu, H. et al., J. Neurosci. Res., 2004, 76: 713-722]. Of note, Iₕ was found to increase in medium and/or large-size DRG neurons after peripheral nerve injuries. Several studies have revealed that Iₕ has an important role in mechanical allodynia in neuropathic pain conditions [Dunlop, J. et al., Curr. Pharm. Des., 2009, 15: 1767-1772; Jiang, Y. Q. et al., Neurochem. Res., 2008, 33: 1979-1989]. Chaplan et al. suggested the importance of HCN channels in both touch-related pain and spontaneous neuronal discharge originating in the damaged DRG [Chaplan, S. R. et al., J. NeuroSci., 2003, 23: 1169-1178], and Takasu et al. reported that spinal HCN channels, most likely at primary afferent terminals, contribute to the maintenance of chronic pain [Takasu, K. et al., Pain, 2010, 151: 87-96]. Consistent with the previous studies of the present inventors [Park, C. K. et al., Pain, 2009, 144: 84-94], they found that Iₕ is more preferentially important for mechanical allodynia than thermal hyperalgesia. A specific blocker of Iₕ, ZD7288, reversed both pain behavior and the spontaneous discharges in injured nerve fibers [Chaplan, S. R. et al., J. NeuroSci., 2003, 23: 1169-1178]. These results suggest that pharmacological blockade of Iₕ could have therapeutic potential under neuropathic conditions. Meanwhile, drugs capable of effectively blocking Iₕ have not been developed yet.

Eugenol is a clear to pale yellow oily liquid extracted from clove oil, nutmeg, cinnamon, basil and bay leaf. It is slightly soluble in water and soluble in organic solvents. It has a clove-like aroma, but an unpleasant smell at high concentrations. The name is derived from the scientific name for clove, Eugenia aromaticum or Eugenia caryophyllata. Eugenol is the main component in the essential oil extracted from cloves, comprising 72 to 90% of the total. Eugenol is commonly used as local antiseptics and anesthetics, and has restorative and prosthodontic applications in dentistry by combination with zinc oxide, because of its ability to allay tooth pain most commonly caused by noxious thermal stimuli [Markowitz, K. et al., Oral Surg. Oral Med. Oral Pathol., 1992, 73: 729-737]. However, since eugenol is hepatotoxic, overdose may cause a wide range of symptoms such as blood in the patient's urine, convulsions, diarrhea, nausea, unconsciousness, dizziness, or rapid heartbeat. The present inventors have published several papers regarding eugenol [Chung, G. et al., J. Dent. Res., 2008, 87: 137-141; Lee, M. H. et al., J. Dent. Res., 2005, 84: 848-851; Park, C. K. et al., J. Dent. Res., 2006, 85: 900-904; Park, C. K. et al., Pain, 2009, 144: 84-94].

### SUMMARY OF THE INVENTION

An object of the present invention is to provide a method for treating mechanical allodynia comprising administering to a patient a therapeutically effective amount of eugenol or a pharmaceutically acceptable salt thereof, wherein the concentration of the administered eugenol or a pharmaceutically acceptable salt thereof is lower than that which would inhibit voltage-gated calcium channels (VGCCs) and voltage-gated sodium channels (VGSCs).

Another object of the present invention is to provide a method for blocking hyperpolarization-activated current (Ih) in neurons comprising administering to a patient a therapeutically effective amount of eugenol or a pharmaceutically acceptable salt thereof, wherein the concentration of the administered eugenol or a pharmaceutically acceptable salt thereof is lower than that which would inhibit VGSCs.

Still another object of the present invention is to ameliorate mechanical allodynia in neuropathic pain patients by providing eugenol at a concentration lower than that at which it inhibits VGCCs and VGSCs to show its analgesic effects, more particularly, to block Iₕ by providing eugenol at the above lower concentration which selectively inhibits HCN channels.

Still another object of the present invention is to provide a transdermal preparation for treating mechanical allodynia comprising the composition.

Still another object of the present invention is to provide a use of eugenol in other pathological pain conditions, including tooth pain for one or more of the above objects.

### DESCRIPTION OF DRAWINGS

FIG. 1 shows normalized Iₕ with −100 mV command potential from a holding potential (Vₕ) of −50 mV in medium- to large-size neurons.

FIG. 2 shows the effects of eugenol on Iₕ in TG neurons (trigeminal ganglion neurons); (A) The left figure represents long time-base recordings of Iₕ measured during 2 s hyperpolarizing test potential of −100 mV from a holding potential of −50 mV with 15 s interval. Eugenol (200 μM) was applied during the time indicated by the horizontal bar. The right figure represents fast time-base recordings of superimposed Iₕ evoked by test pulse at the points indicated in the left panel. (B) The left figure represents a summary of the effects of eugenol on Iₕ in neonatal and adult TG neurons. The right figure represents recordings of Iₕ after the extracellular application of eugenol vs control trace. (C) represents voltage responses to depolarizing and hyperpolarizing current injection (n=4). The inset represents superimposed Iₕ traces before and during exposure to eugenol (200 μM).

FIG. 3 shows the concentration-response relationship of Iₕ inhibition by eugenol; the top figure represents the concentration-response relationship, the bottom figure represents recording of superimposed Iₕ before and during exposure to eugenol at each concentration, and eugenol (500 μM and 1 mM) produced complete Iₕ block.

FIG. 4 shows the effects of eugenol on firing patterns in TG neurons; (A) the left figure represents traces after a 0.5-s current injection (50 pA) before and during exposure to eugenol (200 μM), and the Right figure represents summary for eugenol-induced firing inhibition. (B) The left figure represents the comparison of action potentials at an expanded time scale before and after exposure to eugenol (200 μM), and the Right figure represents a summary of the dV/dt analysis. (C) The left figure represents superimposed current traces before and during exposure to eugenol (200 μM) in the normal bath solution where inward currents result from VGSCs, and the right figure represents a summary of eugenol-induced VGSC currents inhibition (n=3).

FIG. 5 shows the current-voltage relationship and effects of eugenol on reversal potential; (A) The left figure represents Iₕ evoked by hyperpolarizing test pulses of −50 to −120 mV in −10 mV increments from Vₕ of −50 mV (top), and Iₕ superimposed before and during exposure to eugenol (200 μM), and recovery. The right figure represents current-voltage relationships measured at the end of the pulse (▾). (B) The left figure represents the reversal potential determined by applying a −120 mV hyperpolarizing prepulse for 1 s then depolarizing in 10 mV increments from −110 mV to −50 mV (top), and superimposed Iₕ before and during exposure to eugenol (200 μM) (bottom). The right figure represents mean instantaneous Iₕ (↓) plotted with respect to voltage with a linear regression performed.

FIG. 6 shows the mechanism for the inhibitory action of eugenol on Iₕ; (A) the left figure represents long time-base recordings of Iₕ measured during exposure to eugenol (200 μM) with the pretreatment of NEM (50 μM) (top). Superimposed fast time-base recordings are shown in the bottom. The Right figure represents a summary of the effects of NEM on eugenol-induced Iₕ inhibition. (B) represents a summary of the effects of the membrane permeable cAMP analogue 8-Br-cAMP (100 μM), and cAMP isoforms Sp-cAMPs (100 μM) and Rp-cAMPs (100 μM) on eugenol (200 μM)-induced Iₕ inhibition. The inset represents a recording of Iₕ after the extracellular application of eugenol in the presence of 8-Br-cAMP in the pipette solution. (C) represents summary for the concentration-response effect of 8-Br-cAMP on eugenol (200 μM)-induced Iₕ inhibition.

FIG. 7 shows the effects of eugenol on 4 in a trigeminal neuropathic pain model. (A) The left figure represents photographs of DiI-label TG neurons (red, DiC filter) (a), differential interference contrast (DIC) (b), and merged image (c). TG neurons were labeled after DiI application to the proximal end of the transected infraorbital nerve 7 days after ION-CCI (a chronic constriction injury of the infraorbital nerve). The middle figure represents recordings of superimposed Iₕ before and during exposure to eugenol (200 μM). The right figure represents a summary of eugenol-induced Iₕ inhibition in TG neurons from control and ION-CCI rats. (B) represents the effects of eugenol on mechanical allodynia and thermal hyperalgesia in ION-CCI rats. (Ba) represents the effects of eugenol injected subcutaneously on mechanical allodynia in rats with 7 day ION-CCI. Vehicle had no effect upon the mechanical allodynia produced by ION-CCI. Subcutaneous injection of eugenol (1 μg, 5 μg, 10 μg) significantly produced prolonged the antiallodynic effects (in each group, n=6, *p<0.05). (Bb) represents the effects of eugenol injected subcutaneously on thermal hypersensitivity in rats. Vehicle or a low dose of eugenol (10 μg) had no effect upon the thermal hypersensitivity. High doses of eugenol (50 μg) significantly inhibited thermal hypersensitivity (in each group, n=6, *p<0.05). Note the difference in the range of eugenol concentrations used for mechanical allodynia and thermal hyperalgesia.

### DETAILED DESCRIPTION OF THE PREFERRED EMBODIMENTS

In one aspect to achieve the above objects, the present invention provides a pharmaceutical composition for treating mechanical allodynia, which provides eugenol or a pharmaceutically acceptable salt thereof at a concentration lower than that which would inhibit voltage-gated sodium channels (VGSCs).

The compound of the present invention, “eugenol” is a clear to pale yellow oily liquid extracted from clove oil, nutmeg, cinnamon, basil and bay leaf, and is the main component in the essential oil extracted from cloves, comprising approximately 72 to 90% of the total. Eugenol has a structure of the following Chemical Formula 1, and is also called 4-allyl-2-methoxyphenol. Eugenol is commonly used as a local antiseptic and anesthetic, and has restorative and prosthodontic applications in dentistry. However, since eugenol is hepatotoxic, overdose may cause a wide range of symptoms such as blood in the patient's urine, convulsions, diarrhea, nausea, unconsciousness, dizziness, or rapid heartbeat.

In the present invention, when eugenol is used at a concentration lower than that at which it inhibits VGCCs and/or VGSCs to show its analgesic effects, the most severe neuropathic pain, mechanical allodynia, can be selectively alleviated while minimizing its toxicity and unpleasant smell. Eugenol has been used as a dental anesthetic for many years, and thus is commercially available. Alternatively, it can be extracted and purified from cloves or chemically synthesized according to a method known in the art.

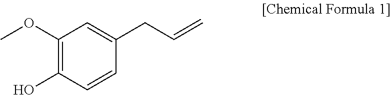

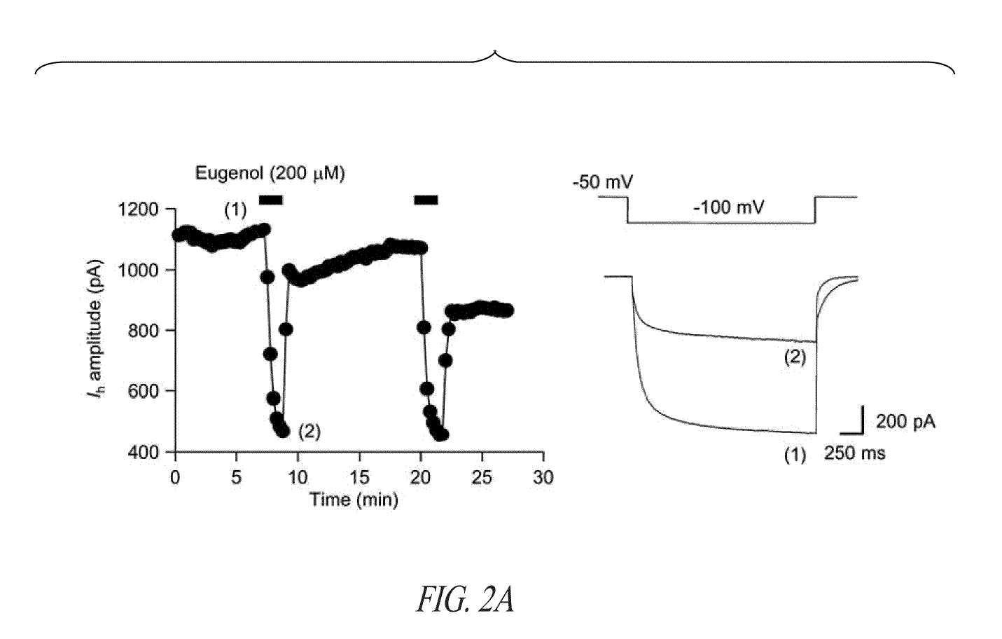

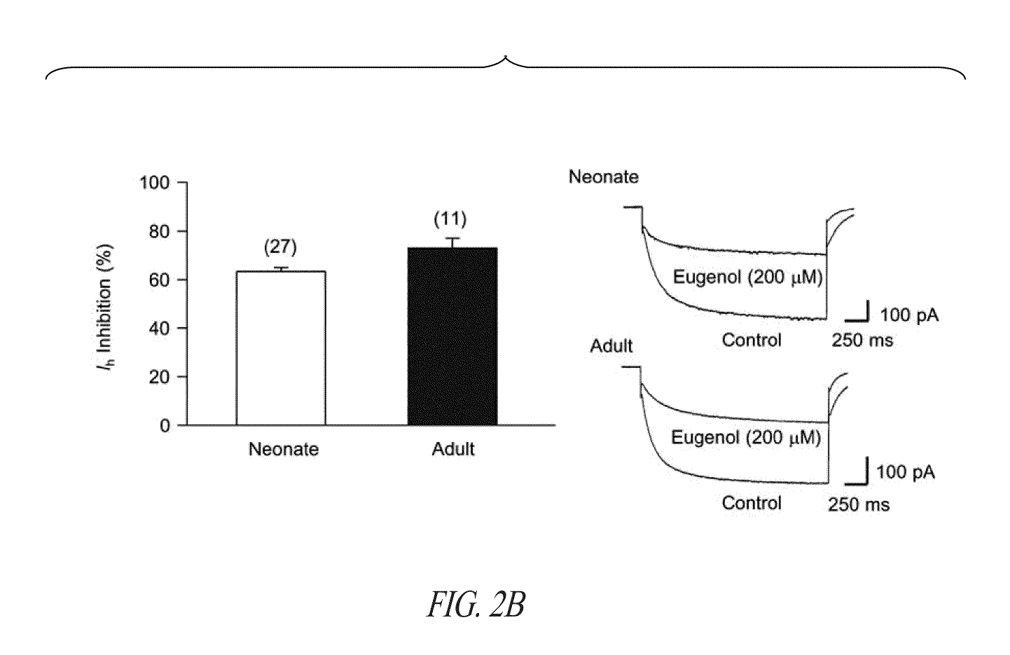

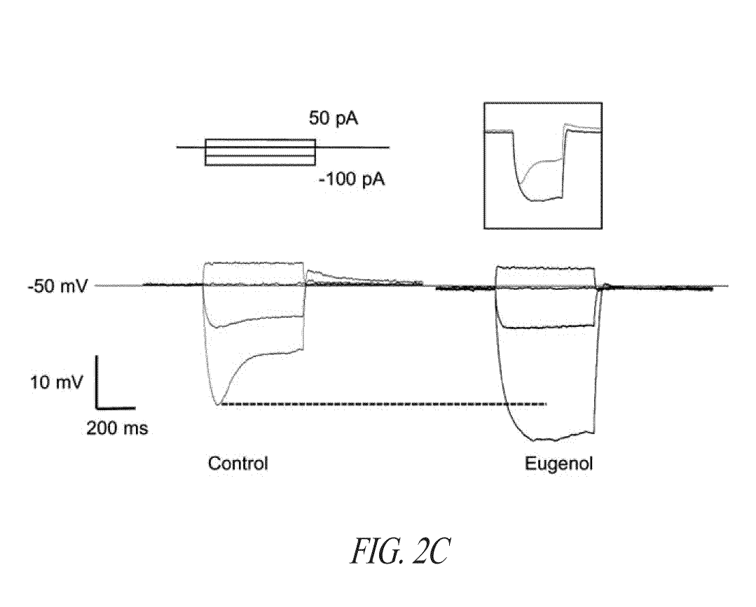

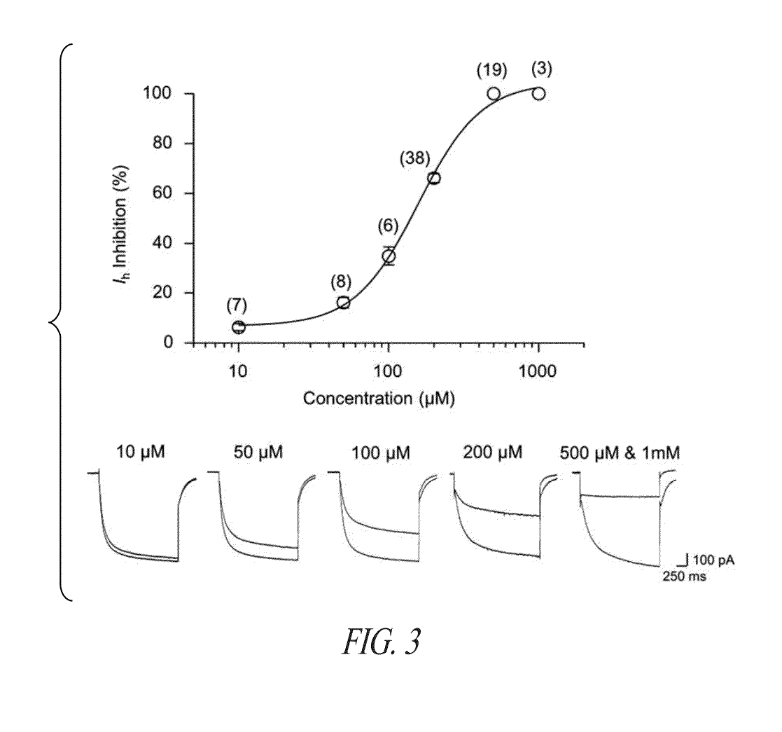

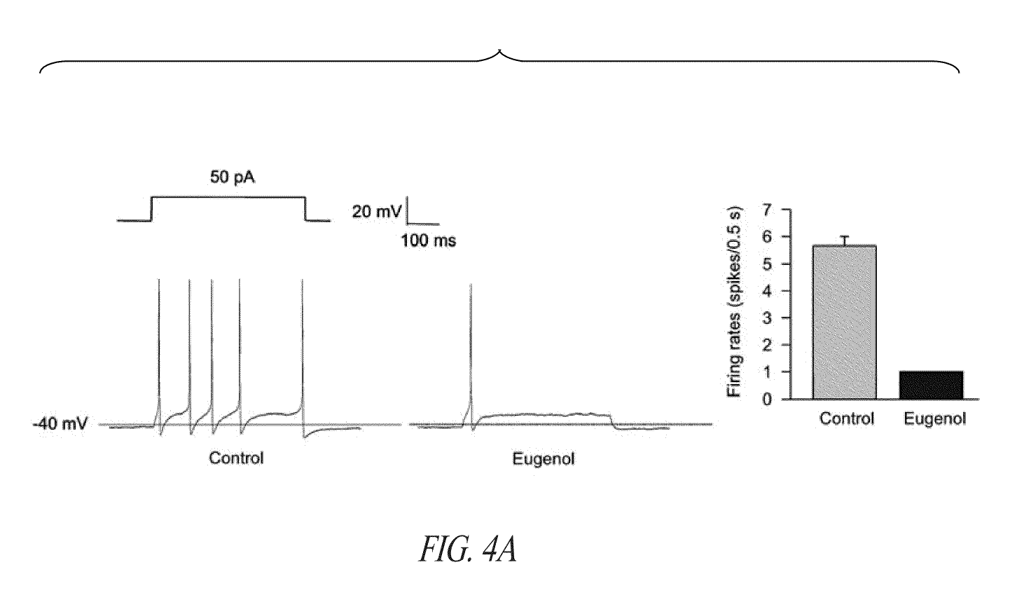

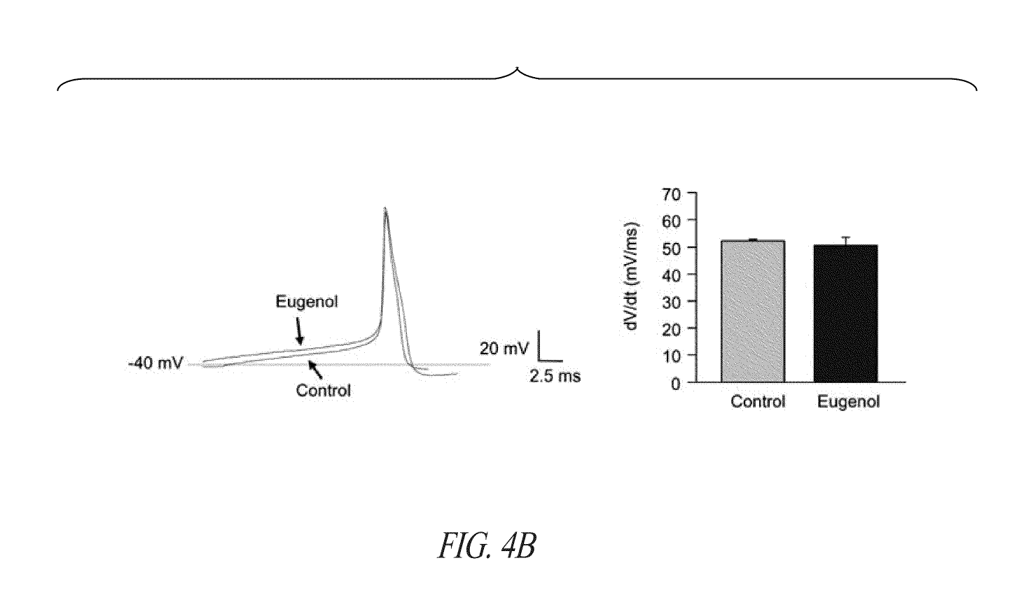

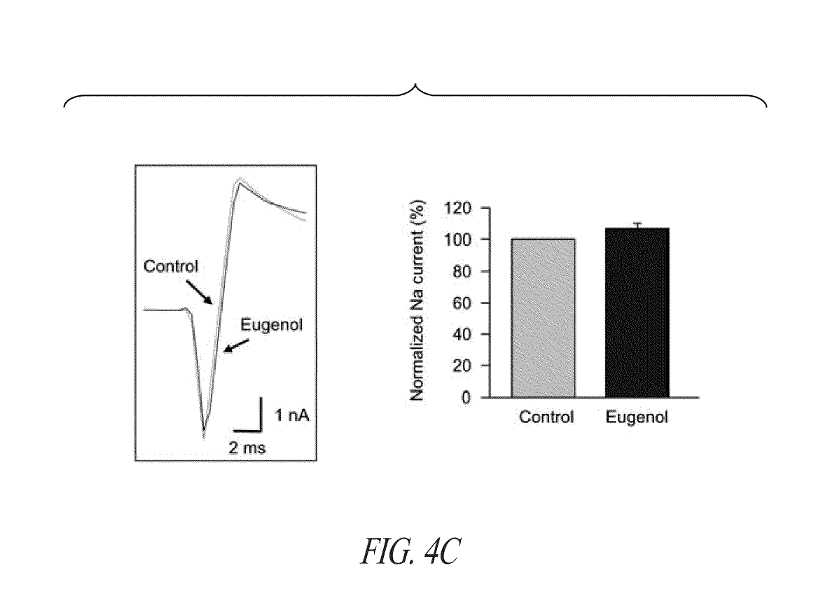

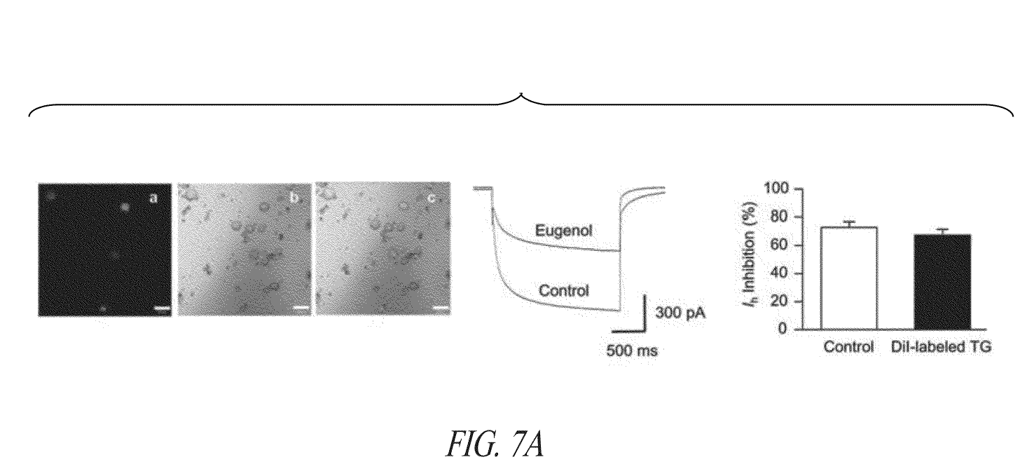

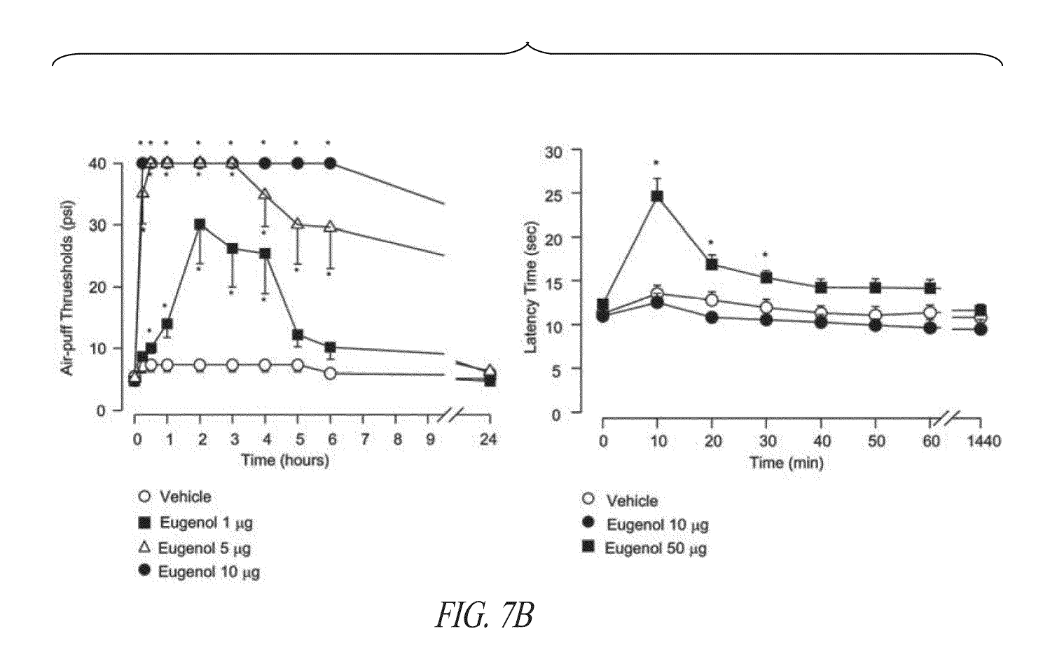

According to one embodiment of the present invention, eugenol (200 μM), at a concentration lower than that at which it inhibits VGSCs (10 mM), effectively inhibited hyperpolarization-activated current A acting via the selective inhibition of HCN channels. Its inhibitory effect was reversibly and repetitively reproduced by sequential application. In addition, the inhibitory effect in neonatal rats was comparable with that in adult rats (FIG. 2). The inhibitory effect was dose dependent, and IC₅₀ was 157 μM. Eugenol effectively inhibited Iₕ between 50 μM and 1 mM (FIG. 3). The firing patterns before and during exposure to 200 μM eugenol of the present invention showed that VGSCs were not affected by 200 μM eugenol (FIG. 4). In a trigeminal neuropathic pain model, when eugenol is administered at a concentration lower than that which would inhibit VGSCs (50 μg; 10 mM), it showed an ameliorating effect on mechanical allodynia, but no effect on thermal hyperalgesia (FIG. 7). A previous study showed that a high dose of eugenol (>50 μg) ameliorated thermal hyperalgesia via inhibition of VGSCs. Thus, it was confirmed that eugenol, at a concentration lower than that at which it inhibits VGSCs, selectively ameliorated mechanical allodynia independent of VGSCs.

As used herein, the term “pharmaceutically acceptable salt” refers to salts that retain the desired biological and/or physiological activities of the above compound and exhibit minimal undesired toxicological effects. An acid addition salt formed by a pharmaceutically acceptable free acid is useful as a salt. The acid addition salts may be prepared by a conventional method, for example, by dissolving the compound in an excessive amount of acid aqueous solution, and then precipitating the salt using a water-miscible organic solvent such as methanol, ethanol, acetone or acetonitrile. Acid or alcohol (e.g., glycol monomethyl ether) in a molar amount equal to the compound and water is heated, and the mixture is dried by evaporation or the precipitated salt can be suction-filtered. At this time, as the free acids, organic acids and inorganic acids may be used. Examples of the inorganic acids may include hydrochloric acid, hydrobromic acid, phosphoric acid, nitric acid, sulfuric acid, and tartaric acid, and examples of the organic acids may include methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, and hydroiodic acid, but are not limited thereto.

Further, a pharmaceutically acceptable metal salt may be prepared using a base. An alkali metal salt or alkaline earth metal salt may be obtained by a method, for example, by dissolving a compound in an excessive amount of alkali metal hydroxide or alkaline earth metal hydroxide solution, filtering the undissolved salt, and then evaporating and drying the filtrate. In the case of metal salts, a sodium, potassium, or calcium salt is pharmaceutically preferable, but it is not limited thereto. The corresponding silver salt may be obtained by reacting alkali metal salt or alkaline earth metal salt with a suitable silver salt (e.g. silver nitrate).

The pharmaceutically acceptable salt of eugenol includes salts of acidic or basic groups, which can be present in eugenol, unless otherwise specifically indicated. For example, the pharmaceutically acceptable salt includes sodium salt, calcium salt, and potassium salt of hydroxy group, and the other pharmaceutically acceptable salts of the amino group include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate). Further, the salts can be prepared by a preparation method known in the related art.

As used herein, the term “concentration lower than that which would inhibit VGSCs” means a concentration of eugenol lower than that at which it inhibits VGSCs present in nerves, preferably, in peripheral nerves, and may be 80%, 70%, 60%, 50%, 40%, 30%, 20% or 10% lower than the concentration inhibiting VGSCs. Conventionally, eugenol was used at a concentration of more than 50 μg, namely, 10 mM in order to inhibit VGSCs. The present inventors were first to demonstrate that eugenol, at a concentration lower than that at which it inhibits VGSCs as an analgesic mechanism, selectively inhibits HCN channels present in nerves, preferably, in peripheral nerves so as to block A generated from HCN, thereby treating mechanical allodynia more sensitive to Iₕ. Preferably, the composition of the present invention may be prepared to provide eugenol at a concentration of less than 2 mM, and more preferably, 1 mM or less, or 200 μM or less (FIG. 7B), 50 μM to 2 mM, 50 μM to 1 mM, 200 μM to 2 mM, or 50 μM to 1 mM. These concentrations may be the concentrations lower than that which would inhibit VGSCs and VGCCs, and may be 80%, 70%, 60%, 50%, 40%, 30%, 20% or 10% lower than the concentration inhibiting VGSCs and VGCCs.

In one aspect of the present invention, mechanical allodynia can be treated by providing eugenol at a concentration lower than the conventional concentration used for analgesic effect on tooth pain, other pathological pain conditions, or neuropathic pain conditions, and is preferably 80%, 70%, 60%, 50%, 40%, 30%, 20% or 10% lower than the conventional concentration of eugenol.

As used herein, the term “neuropathic pain” is a common type of chronic, non-malignant pain, which is the result of an injury or malfunction in the peripheral or central nervous system and serves no protective biological function. It may occur, for example, due to trauma, surgery, herniation of an intervertebral disk, spinal cord injury, diabetes, infection with herpes zoster, HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs. In contrast to nociceptive pain, neuropathic pain is frequently described as “burning,” “electric,” “tingling,” or “shooting” in nature. It is often characterized by allodynia defined as pain resulting from a stimulus that does not ordinarily elicit a painful response such as light touch, and hyperalgesia defined as an increased sensitivity to a normally painful stimulus, and may persist for months or years beyond the apparent healing of any damaged tissues.

As used herein, the term “mechanical allodynia” refers to the abnormal perception of pain from usually light mechanical stimulation, among allodynia which occurs due to a non-noxious stimulus that does not normally provoke pain, and it is the most severe neuropathic pain.

The composition of the present invention can be used without limitation for the treatment of mechanical allodynia among traumatic or injurious pain such as postsurgical pain; metabolic pain such as diabetic neuropathy; ischaemic or hemorrhagic pain such as central pain after stroke; toxic pain such as heavy metal poisoning or chemotherapy; compression pain such as spinal stenosis or carpal tunnel syndrome; immune-mediated pain such as multiple sclerosis; inflammatory pain such as post-herpetic neuralgia and hereditary pain such as Fabry's disease. It can be also used for the treatment of mechanical allodynia in the orofacial area. In one aspect, the present invention provides a method for treating mechanical allodynia comprising administering to a patient a therapeutically effective amount of eugenol or a pharmaceutically acceptable salt thereof, wherein the concentration of the administered eugenol or a pharmaceutically acceptable salt thereof is lower than that which would inhibit voltage-gated sodium channels (VGSCs).

In another aspect, the present invention provides a pharmaceutical composition for blocking Iₕ, which provides eugenol or a pharmaceutically acceptable salt thereof at a concentration lower than which would inhibit VGSCs.

The composition of the present invention directly blocks Iₕ of nerves, preferably, peripheral nerves. As used herein, the term “hyperpolarization-activated current (Iₕ)” means a current generated by hyperpolarization-activated cyclic nucleotide-gated (HCN) ion channels. It was first designated as a pacemaker current, which contributes to the rate of rhythmic oscillations in cardiac cells, and also in cranial nerve cells. In particular, it has been also identified in DRG neurons, where most ectopic discharges occur after peripheral nerve injury. The HCN channels are ion channels distributed in cardiac and nerve cells. In general, hyperpolarization of the nerve cell membrane could activate and open HCN channels, leading to the influx of extracellular cations into the cytoplasm, and consequently, membrane depolarization occurs to produce a current. Cyclic nucleotide-binding domain (CNBD) present in HCN channels binds with cyclic nucleotides such as cAMP, so as to promote channel opening and voltage dependence. In one aspect, the present invention provides a method for blocking hyperpolarization-activated current (Ih) in neurons comprising administering to a patient a therapeutically effective amount of eugenol or a pharmaceutically acceptable salt thereof, wherein the concentration of the administered eugenol or a pharmaceutically acceptable salt thereof is lower than that which would inhibit voltage-gated sodium channels (VGSCs).

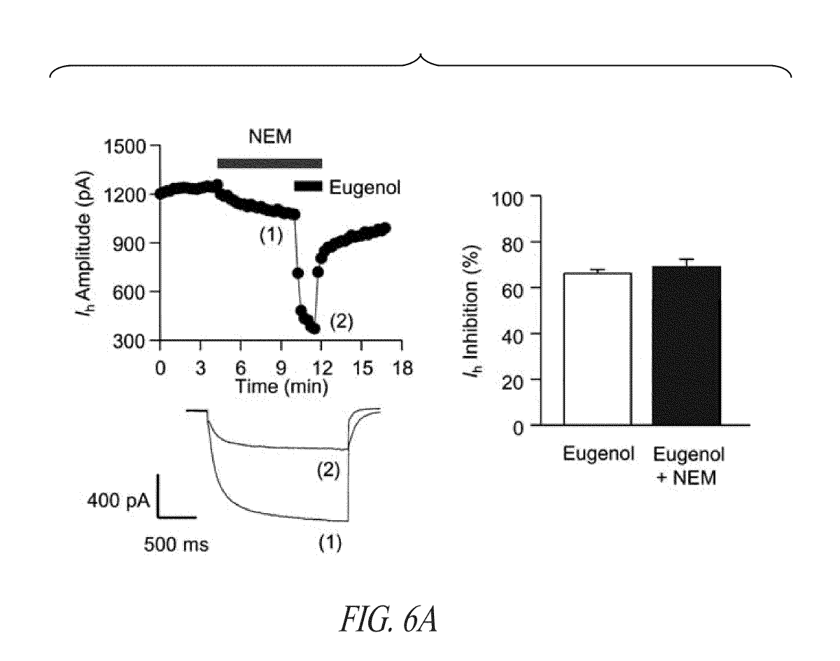

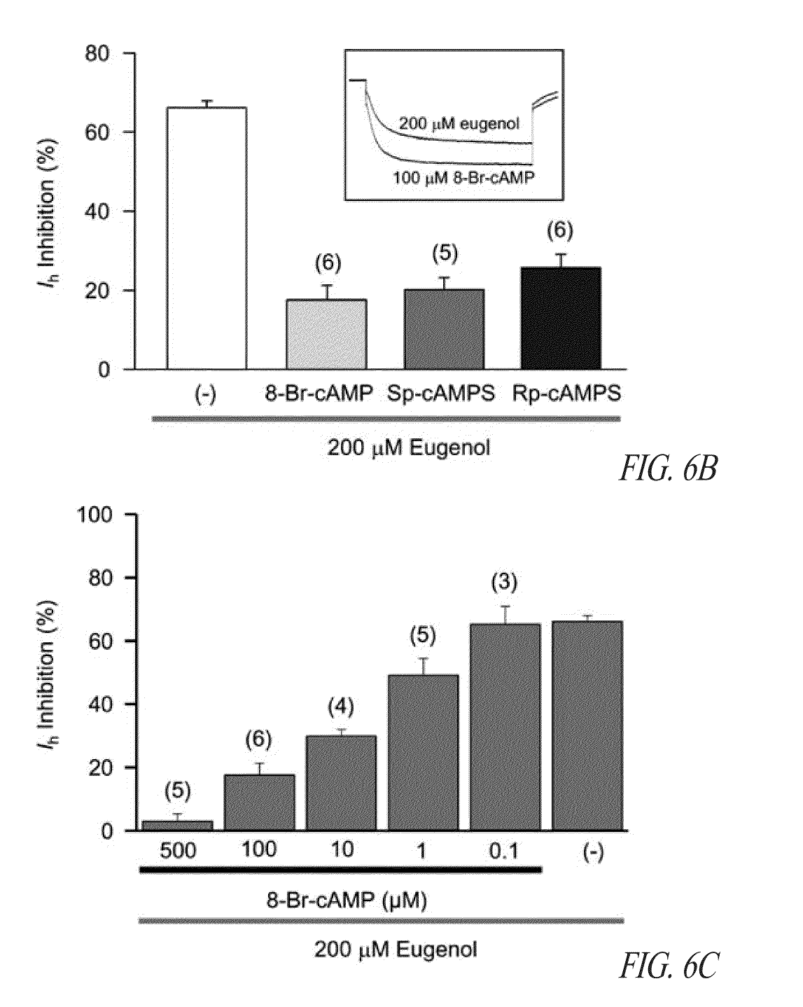

According to an embodiment of the present invention, in order to confirm the mechanism for the inhibitory action of eugenol on Iₕ, it was examined whether coupling with G-protein participates in the inhibitory effect of eugenol on Iₕ by using NEM (N-ethylmaleimide), a sulpho-alkylating agent which is known to block coupling of G-protein coupled receptor (GPCR). As a result, no effect was observed, indicating that the eugenol-induced Iₕ inhibition is unlikely to be associated with GPCR (FIG. 6A). Its association with the cyclic nucleotides such as cAMP, known to directly modulate HCN channel gating, was also examined. Treatment of the cell-permeable cAMP analogue 8-Br-cAMP, and the cAMP isoforms, Sp-cAMP and Rp-cAMP significantly attenuated the eugenol-induced Iₕ inhibition (FIG. 6B), which was reversely proportional to the intracellular concentration of cAMP (FIG. 6C). These results suggest that eugenol shares CNBD of HCN channels with cAMP, although the inhibitory action of eugenol on Iₕ might be independent of the modulatory action or downstream pathways of cAMP. While cAMP increases activation kinetics of Iₕ, eugenol decreases overall amplitude of Iₕ. As a consequence, the effect of cAMP is more clearly seen at an earlier point in time, while that of eugenol is uniform until the steady state time point. In addition, eugenol inhibited A more than the portion enhanced by cAMP. This is in contrast to the cAMP-dependent inhibition of Iₕ by morphine, in which only the cAMP-enhanced portion was inhibited.

Iₕ was found to increase in medium and/or large-size DRG neurons after peripheral nerve injuries. Several studies have revealed that Iₕ has an important role in mechanical allodynia in neuropathic pain conditions [Dunlop, J. et al., Curr. Pharm. Des., 2009, 15: 1767-1772; Jiang, Y. Q. et al., Neurochem. Res., 2008, 33: 1979-1989]. Chaplan et al. suggested the importance of HCN channels in both touch-related pain and spontaneous neuronal discharge originating in the damaged DRG [Chaplan, S. R. et al., J. NeuroSci., 2003, 23: 1169-1178], and Takasu et al. reported that spinal HCN channels, most likely at primary afferent terminals, contribute to the maintenance of chronic pain [Takasu, K. et al., Pain, 2010, 151: 87-96]. Consistent with the previous studies of the present inventors [Park, C. K. et al., Pain, 2009, 144: 84-94], they found that Iₕ is more preferentially important for mechanical allodynia than thermal hyperalgesia. A specific blocker of Iₕ, ZD7288, reversed both pain behavior and the spontaneous discharges in injured nerve fibers [Chaplan, S. R. et al., J. NeuroSci., 2003, 23: 1169-1178]. These results suggest that pharmacological blockade of Iₕ could have a therapeutic potential under neuropathic conditions. In an embodiment of the present invention, it was found that the inhibitory effect of eugenol on Iₕ in TG neurons was dose dependent (FIG. 3), and eugenol reduces the excitability of TG neurons and may diminish activity at central terminals to the medullary dorsal horn, the counterpart of the spinal dorsal horn in the spinal system (FIG. 4A). In a trigeminal neuropathic pain model ION-CCI rat, the inhibition of Iₕ by 200 μM of eugenol was indistinguishable between the injured TG neurons and the naive TG neurons, suggesting that eugenol could be useful for ameliorating mechanical allodynia in a trigeminal neuropathic pain condition (FIG. 7A). In a head withdrawal reflex test, it was found that eugenol significantly ameliorated mechanical allodynia at a lower range of concentrations than those which were effective for ameliorating thermal hyperalgesia (FIG. 7B).

In one aspect of the present invention, Iₕ can be blocked by providing eugenol at a concentration lower than that which would inhibit VGSCs, that is, at 80%, 70%, 60%, 50%, 40%, 30%, 20% or 10% lower than the concentration inhibiting VGSCs. These concentrations may be the concentrations lower than that which would inhibit VGSCs and VGCCs, and may be 80%, 70%, 60%, 50%, 40%, 30%, 20% or 10% lower than the concentration of inhibiting VGSCs and VGCCs. In one aspect of the present invention, Iₕ can be blocked by providing eugenol at a concentration lower than the conventional concentration used for analgesic effect on tooth pain, other pathological pain conditions, or neuropathic pain conditions, and preferably at 80%, 70%, 60%, 50%, 40%, 30%, 20% or 10% lower than the conventional concentration of eugenol.

In still another aspect, eugenol or a pharmaceutically acceptable salt thereof can be provided to be formulated as a transdermal preparation for treating mechanical allodynia comprising the composition.

The method according to claim 1, wherein is formulated as a transdermal preparation.

As used herein, the term “transdermal preparation” is a formulation which exhibits its efficacy by passage of a drug through the skin, and is formulated into an externally applicable formulation or an externally attachable formulation. Upon transdermal administration, skin permeation of active ingredient occurs by intercellular diffusion, transcellular diffusion, or diffusion through appendages such as hair follicles and sweat ducts according to chemical potential, namely, concentration gradient. It is disadvantageous in that it is difficult to permeate undamaged skin, but it is beneficial in terms of drug efficiency, control of permeation rate, and direct application to the wound lesion. In addition, it has the advantages of relatively constant blood level, minimization of gastrointestinal side effects, and reduction of liver damage. In order to facilitate skin permeation of the active ingredient, a skin permeation enhancer may be further included in the preparation. Transdermal delivery of the eugenol of the present invention is favorable due to its hydrophobicity.

The transdermal preparation of the present invention is not limited, as long as the preparation can be directly applicable to the surface of a dermal lesion, and for example may be, ointments, creams, gels, lotions, liquids, emulsions, suspensions, plasters (sticks), pastes, liniments, cataplasms, tapes, aerosols, or external powders. Proper carriers, excipients, and diluents typically used in the preparation of transdermal formulations may be further included. The composition is sterile or aseptic, and such a solution is sterile or aseptic and may include water, buffers, isotonic agents or other ingredients known to those of skill in the art that would cause no allergic or other harmful reaction when administered to an animal or human subject.

The ointments, the creams, the gels and the lotions may include a base such as white petrolatum, yellow petrolatum, lanolin, white beeswax, cetanol, stearyl alcohol, stearic acid, hydrogenated oil, hydrocarbon gel, polyethylene glycol, liquid paraffin, squalane, etc.; a solvent or a dissolving agent such as oleic acid, isopropyl myristate, glyceryl triisooctanoate, crotamiton, diethyl sebacate, disopropyl adipate, hexyl laurate, a fatty acid, a fatty acid ester, an aliphatic alcohol, a vegetable oil, etc.; an antioxidant such as a tocopherol derivative, L-ascorbic acid, dibutylhydroxytoluene, butylhydroxyanisole, etc.; an antiseptic such as parahydroxybenzoate ester, etc.; a humectant agent such as glycerin, propylene glycol, sodium hyaluronate, etc.; a surfactant such as a polyoxyethylene derivative, a glyceryl fatty acid ester, a sucrose fatty acid ester, a sorbitan fatty acid ester, a propylene glycol fatty acid ester, lecithin, etc.; a thickener such as carboxyvinylpolymer, xanthan gum, carboxymethylcellulose, sodium carboxymethylcellulose salt, hydroxypropyl cellulose, hydroxypropyl methylcellulose, etc. If desired, a stabilizing agent, a preservative, an absorption promoting agent, a pH adjuster, or other suitable additives may be blended.

The solutions or emulsions may include carriers such as solvents, solubilizers or emulsifiers, for example water, ethanol, isopropanol, ethyl carbonate, ethyl acetate, benzyl alcohol, benzyl benzoate, propylene glycol, 1,3-butyl glycol oil, glycerol fatty acid ester, polyethylene glycol, and fatty acid esters of sorbitan.

The suspensions may include carriers such as liquid diluents, for example, water, ethanol or propylene glycol; suspending agents, for example, ethoxylated isostearyl alcohols, polyoxyethylene sorbitol esters, and polyoxyethylene sorbitan esters; microcrystalline cellulose, aluminium metahydroxide, bentonite, agar, or tragacanth.

The plasters may be formulated using lead monoxide, olive oil, and lard, the pastes may be formulated using fine powder of zinc oxide, salicylic acid, starch, and petrolatum, and the liniments may be formulated using camphor oil, olive oil, methyl salicylate, tragacanth, sodium carboxymethylcellulose, glycerin and zinc oxide.

The cataplasms may include a tackifier such as polyacrylic acid, polyacrylic acid copolymer, etc.; a crosslinking agent such as aluminum sulfate, aluminum potassium sulfate, aluminum chloride, magnesium alminometasilicate, dihydroxyalminum acetate, etc.; a thickener such as sodium polyacrylate, polyvinyl alcohol, polyvinylpyrrolidone, gelatin, sodium alginate, carboxymethylcellulose, carboxymethycellulose sodium salt, hydroxypropyl cellulose, hydroxypropyl methylcellulose, etc.; a polyvalent alcohol such as glycerin, polyethylene glycol (macrogol), propylene glycol, 1,3-butandiol, etc.; a surfactant such as polyoxyethylene derivative, etc.; a flavor such as 1-menthol, etc.; an antiseptic such as parahydroxybenzoate ester, etc.; purified water. If desired, a stabilizer, a preservative, an absorption promoter, a pH adjuster or other suitable additives may be blended.

The tapes may include an adhesive agent such as styrene-isoprene-styrene block copolymer (SIS block copolymer), acryl resin, etc.; a tackifier resin such as alicyclic saturated hydrocarbon resin, rosin resin, terpene resin, etc.; a softener such as liquid rubber, liquid paraffin, etc.; an antioxidant such as dibutylhydroxytoluene, etc.; a polyvalent alcohol such as propylene glycol, etc.; an absorption promoter such as oleic acid, etc.; a surfactant such as a polyoxyethylene derivative, etc.; or a suitable additive. The aqueous tapes may be also prepared by blending an aqueous high molecular compound such as sodium polyacrylate or polyvinyl alcohol with a small amount of purified water. In this case, a stabilizer, a preservative, an absorption promoter, a pH adjuster or other suitable additives may be also blended, if desired.

The aerosols may include the ingredients used in ointments, creams, gels, suspensions, emulsions, solutions, and lotions, namely, a base such as white petrolatum, yellow petrolatum, lanolin, white beeswax, cetanol, stearyl alcohol, stearic acid, dehydrogenated oil, hydrocarbon gel, polyethylene glycol, liquid paraffin, squalane, etc.; a solvent or a dissolving agent such as oleic acid, isopropyl myristate, diisopropyl adipate, isopropyl sevacate, glyceryl triisooctanoate, crotamiton, diethyl sevacate, hexyl laurate, a fatty acid, a fatty acid ester, an aliphatic alcohol, a vegetable oil, etc.; an antioxidant such as a tocopherol derivative, L-ascorbic acid, dibutylhydroxytoluene, butylhydroxyanisole, etc.; an antiseptic such as parahydroxybenzoate ester, etc.; a humidity preserving agent such as glycerin, propylene glycol, sodium hyaluronate etc.; a surfactant such as a polyoxyethylene derivative, a glycerin fatty acid ester, a sucrose fatty acid ester, a sorbitan fatty acid ester, a propylene glycol fatty acid ester, lecithin, etc.; a thickener such as carboxyvinyl polymer, xanthan gum, carboxymethylcellulose, sodium carboxymethylcellulose, hydroxypropyl cellulose, hydroxypropyl methylcellulose, etc. Additionally, a stabilizer, a buffer, a sweetening agent, a suspending agent, an emulsifying agent, a flavor, a preservative, a solubilizing agent, or other suitable additive may also be blended, if desired. In particular, a propellent such as chlorofluorohydrocarbon, propane/butane and dimethyl ether may be further included.

The external powders may include an excipient such as potato starch, rice starch, corn starch, talc, zinc oxide, etc. or a suitable additive. In this case, a stabilizer, a preservative, an absorption promoter, or other suitable additives may also be blended, if desired.

The preparation means for the transdermal formulation of the present invention is not particularly limited, and it may be prepared by kneading each ingredient well and, if necessary, adding a base in accordance with the typical method for transdermal formulations. Further, in the preparation of the cataplasms and tapes, the kneaded mixture is spread on a release paper and dried. Additionally, it is laminated with a flexible backing, and cut in a desired size.

The transdermal preparation of the present invention, for example, ointments, solutions (suspensions, emulsions, lotions, etc.), aerosols, and external powders, is practiced by directly applying it to the wound lesion or by spreading it on the cloth or immersing it to the cloth and then applying it in accordance with the usual application method. In addition, the cataplasms or tapes are also directly applied to the wound lesion.

The suitable administration dosage of the pharmaceutical composition of the present invention may be readily determined by those skilled in the art in accordance with the formulation method, patient's age, body weight, sex, condition of diseases, diet, administration time, administration route, reaction sensitivity, and other factors known to medical practitioners. When used as a formulation for transdermal administration, the administration dosage may be determined considering the transdermal permeation rate, cell absorption or the like. Preferably, it may be applied in an amount of 1 mg/cm² to 50 mg/cm² or 2 mg/cm² to 20 mg/cm² per unit skin area, but is not limited thereto. Within the range of providing eugenol or the pharmaceutically acceptable salt thereof at a concentration lower than that which would inhibit VGSCs and/or VGCCs, it may be determined by those skilled in the art, considering the content of eugenol or pharmaceutically acceptable salt thereof based on the total weight of the composition. The pharmaceutical composition of the present invention may be prepared to include eugenol or the pharmaceutically acceptable salt thereof at a content of 0.01 to 5.0% by weight, 0.1 to 2.0% by weight, or 0.16 to 1.64% by weight, based on the total weight of the composition, but is not limited thereto. Within the range of providing eugenol or the pharmaceutically acceptable salt thereof at a concentration lower than that which would inhibit VGSCs and/or VGCCs, it may be determined by those skilled in the art.

In one embodiment of the present invention, when the composition of the present invention was applied to the wound lesion, the effect occurred immediately, and was maintained for 7 to 8 hours (FIG. 7B).

Hereinafter, the present invention will be described in more detail with reference to Examples. However, these Examples are for illustrative purposes only, and the invention is not intended to be limited thereby.

### Example 1

### Animal and Surgery

All surgical and experimental procedures were reviewed and approved by the Institutional Animal Care and Use Committee at the School of Dentistry, Seoul National University. Animal treatments were performed in strict accordance with the ethical guidelines of the International Association for the Study of Pain for the investigations of experimental pain in conscious animals [Zimmermann, M., Pain, 1983, 16: 109-110], and the National Institute of Health Guide for the Care and Use of Laboratory Animals. Behavioral tests were carried out on male Sprague Dawley rats (OrientBio, Sungnam, Korea) weighing 200-250 g. The rats were maintained in a temperature-controlled room with a 12/12-h light/dark cycle and fed food and water ad libitum. The animals were allowed to habituate to the housing facilities for 1 week before the experiments.

Surgical procedures were performed under 0.5 mL/kg (i.m.) of a mixture of equal volumes of ketamine (100 mg/mL) and xylazine (20 mg/mL) anesthesia. Under anesthesia, a chronic constriction injury of the infraorbital nerve (ION-CCI) was performed from the original description of Imamura et al. [Imamura, Y. et al., Exp. Brain Res., 1997, 116: 97-103]. A 1-cm-long incision was made along the gingivobuccal margin. The incision was begun proximal to the first molar. Approximately 0.5 cm of the ION was separated from the adhering tissue, and 2 ligatures (5-0 chromic gut) were tied loosely around it. The incision was sutured at 3 points with 4-0 silk. The sham operation was performed without ligation of the infraorbital nerve.

### Example 2

### Preparation and Retrograde Labeling of Trigeminal Ganglion (TG) Neurons

TG neurons from 2- to 7-day-old neonatal rats or adult rats (200 to 300 g) were prepared as previously described [Fang, Z. et al., J. Biol. Chem., 2007, 282: 4757-4764]. Briefly, TG neurons prepared in 4° C. Hanks balanced salt solution (Welgene, Daegu, Korea) were incubated in 2 mL Hanks balanced salt solution containing 0.167% trypsin (Invitrogen, Carlsbad, Calif.) at 37° C. for 40 min. The cells were washed in DMEM and triturated with a flame-polished Pasteur pipette to separate cells and remove processes, and subsequently centrifuged (750 RPM, 5 min), resuspended and placed on 0.5 mg/mL poly-L-ornithine (Sigma, St. Louis, Mo.)-coated glass coverslips (12 mm in diameter). The cells were maintained at 37° C. in a 95% O₂/5% CO₂ incubator.

To identify injured neurons in ION-CCI rats, the ION was transected 7 days after ION-CCI and DiI (Molecular Probes, Oreg.), retrograde labeled with a fluorescent dye, was placed onto the proximal end of the nerve (n=4). After 3 days, TG neurons were processed as described above. DiI-labeled neurons were identified under fluorescent microscope and used for whole-cell recordings.

### Example 3

### Electrophysiological Recordings

Whole-cell recordings were made using patch electrodes (4 to 6 MΩ) pulled from borosilicate glass on a Brown-Flaming P-97 horizontal micropipette puller (Sutter Instruments, Novato, Calif.). Voltage- and current-clamp experiments were performed using an HEKA EPC9 amplifier and digitized using ITC16 and Pulse v8.54 software (both from HEKA, Lambrecht/Pfalz, Germany). Signals were filtered at 1 kHz and sampled at 3 kHz. Series resistance was typically less than 20 MΩ and was compensated by about 70 to 80%. Electrical recordings were performed at room temperature. The 3 M KCl agar bridge (Warner Instruments, Hamden, Conn.) is used to avoid high liquid junction potentials. Data were analyzed by Origin 6.0 software.

Extracellular solution containing 140 mM NaCl, 2 mM CaCl₂, 1 mM MgCl₂, 5 mM KCl, 10 mM HEPES, 10 mM D-glucose was adjusted to pH 7.4 with NaOH, 290 to 310 mOsm. In experiments, BaCl₂ was used to eliminate inward rectifier K⁺ currents (I_{KIR}). Pipettes were filled with an intracellular solution containing 136 mM K-gluconate, 10 mM NaCl, 1 mM MgCl₂, 10 mM EGTA, 2 mM Mg-ATP, 0.1 mM Na-GTP adjusted to pH 7.4 with KOH, 290 to 310 mOsm. Cells were continuously perfused with extracellular solution using a gravity-fed perfusion system. Recording was started at least 5 min after obtaining the whole-cell configuration.

Iₕ was elicited by a 2 s pulse from a holding potential of −50 mV to −120 mV in 10 mV steps. Iₕ was identified by its response to a CsCl (2 mM) used as blocker of Iₕ channels. The reversal potential of the eugenol-sensitive currents was determined by measuring the tail currents. In current-clamp experiments, action potential firing was elicited by a series of depolarizing current pulses (500 ms in duration) from −100 to 300 pA in 50 pA step increments.

### Example 4

### Behavioral Tests

On postoperative 7 day, ION-CCI rats were placed in customized observation cages that were then placed in a darkened and noise-free room to conduct behavioral observations. The animals were acclimated for at least 30 min. Mechanical allodynia was determined as previously described [Ahn, D. K. et al., Pain, 2009, 146: 114-120], by observing withdrawal behavior, produced by 10 successive trials of constant air-puff pressure (4-s duration, 10-s intervals) in freely moving rats. The intensity and intervals of the air-puff pressure were controlled with a pneumatic pump module (BH2 system, Harvard Apparatus, Holliston, Mass.). The air puffs were applied through a 26-gauge metal tube (length, 10 cm) located 1 cm from the skin at a 90° angle. Thresholds were determined through the air-puff pressure at which each rat withdrew its head in 50% of the trials. The cutoff pressure for the air puffs was 40 psi. The naive rats did not respond to a pressure of less than 40 psi. After eugenol (1 μg/30 μL, 5 μg/30 μL or 10 μg/30 μL; 200 μM, 1 mM, or 2 mM, respectively) was administered subcutaneously into the vibrissa pad, air-puff thresholds were determined at 15 and 30 min, and at 1, 2, 3, 4, 5, 6, and 24 hr after eugenol injection. In the control group, 30 μL of the vehicle (30% DMSO/saline) was injected subcutaneously.

Thermal hyperalgesia was also determined as previously described [Ahn, D. K. et al., Pain, 2009, 146: 114-120; Park, C. K. et al., Pain, 2009, 144: 84-94]. Each rat was placed in a customized cylinder-type acrylic rodent restrainer (height 40 to 60 mm, length 70 to 120 mm). The restrainer had a hole in the top so that the head could receive thermal stimulation and produce a head withdrawal action. Each restrainer was placed in a darkened and noise-free room, and the animals were habituated for at least 30 min before the start of the experiment. After radiant heat was applied to the midregion of the left vibrissa pad, the head withdrawal latency was determined. The application of heat stimuli was performed using an infrared thermal stimulator (Infrared Diode Laser, LVI-808-10, LVI tech, Seoul, Korea). The power and current of the thermal stimulator were adjusted to 11 W and 18.1 A, respectively. The thermal stimuli produced stable withdrawal latencies of approximately 11 s for a 10-cm distance from the heat source to the vibrissa pad. Each rat received 2 stimuli, and the interstimulus interval for each trial was at least 5 min. A cutoff time of 20 s was used in these experiments to prevent possible tissue damage. After eugenol (10 μg/30 μL or 50 μg/30 μL; 2 mM or 10 mM, respectively) was administered subcutaneously into the vibrissa pad, the latencies of withdrawal responses were determined at 10, 20, 30, 40, 50, and 60 min and 24 h after eugenol injection. In the control group, 30 μL of the vehicle (30% DMSO/saline) was injected subcutaneously. The experimenter was blind to the treatment group in all behavioral experiments.

### Example 5

### Drugs

All chemicals were purchased from Sigma. Eugenol and N-ethylmaleimide (NEM) were added to the extracellular solutions. 8-Bromoadenosine 3′,5′-cyclic monophosphate (sodium salt; 8-Bromo-cAMP) and Rp, or Sp isomers of adenosine 3′,5′-cyclic monophosphorothioate were infused into the cytosol through the patch pipette.

### Example 6

### Statistical Analysis

Statistical analyses of the behavioral data sets were carried out by SPSS software (SPSS, Chicago, Ill.) by a repeated measures ANOVA followed by multiple group comparisons using a Bonferroni post hoc analysis. For the in vitro data sets, unpaired or paired Student's t-test was performed to evaluate the difference using Origin 6.0 (Microcal Software, Northampton, Mass.). Differences were considered to be significant when the P value was less than 0.05. All data are presented as mean±standard error of the mean, and the numbers of cells tested are indicated in parentheses.

### Results

### Experimental Example 1

### Inhibition of Eugenol on I_{b} Currents in TG Neurons in a Dose-Dependent Manner

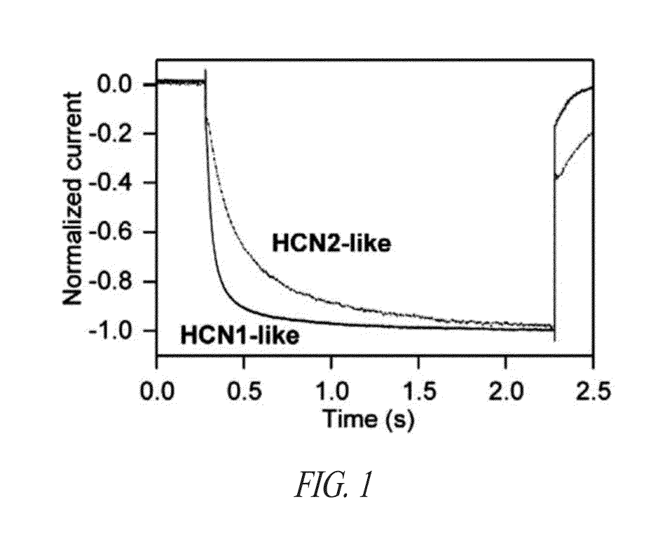

Given that Iₕ has been previously identified in DRG neurons, particularly in medium- and large-size neurons [Scroggs, R. S. et al., J. Neurophysiol., 1994, 71: 271-279], recordings were largely made from TG neurons that ranged 25 to 45 μm in cell diameter. The biophysical properties of Iₕ were analyzed with −100 mV command potential from a holding potential (Vₕ) of −50 mV in these medium- to large-size neurons, Iₕ corresponded to either that of HCN1 or HCN2 (n=54, Table 1 and FIG. 1), based on a previous report [Chen, S. et al., J. Gen. Physiol., 2001, 117: 491-504]. The Iₕ recorded from small-size neurons (<25 μm) was relatively small in amplitude and showed slow kinetics that may suggest the expression of HCN4, as reported by Wan [Wan, Y., Sheng Li Xue Bao, 2008, 60: 579-580]. In the present invention, the effect of eugenol on Iₕ of TG neurons was examined (FIG. 2). Eugenol (200 μM) inhibited Iₕ, and its inhibitory effect was reversible and repetitively reproduced by sequential application (FIG. 2A). The Iₕ inhibition produced by eugenol (200 μM) in neonatal rats (63.3±1.6%, n=27) was comparable with that in adult rats (73.0±3.9%, n=11, P>0.05, FIG. 2B). Thus, neonatal TG neurons were used for the further studies. In current clamp-mode, hyperpolarizing current injection was clearly eliminated by eugenol (200 μM) (FIG. 2C).

Also, the inhibitory effect of eugenol on Iₕ in neonatal TG neurons was dose dependent (FIG. 3). Eugenol effectively inhibited Iₕ between 50 μM and 1 mM (IC₅₀=157 04).

### Experimental Example 2

### Eugenol Decreases Spike Firings in TG Neurons

Iₕ plays an important role in DRG neuronal excitability such as during spontaneous or repetitive activity [Yagi, J. et al., J. Neurophysiol., 1998, 80: 1094-1104]. Indeed, ZD7288, a selective blocker of the Iₕ conductance, prolonged action potential duration, and diminished repetitive firing during tetanic bursts [Hogan, Q. H. and Poroli, M., Brain Res., 2008, 1207: 102-110]. The present inventors tested whether eugenol decreases firing of action potentials in TG neurons. A 50 pA depolarizing current injection induced repetitive firing of action potentials. Firing rates of action potentials were reduced by 200 μM eugenol (n=4, FIG. 4A). The persistence of the first action potential in the presence of eugenol indicated that VGSCs were not affected by 200 μM eugenol. It was found that dV/dt was not changed by 200 μM eugenol application and with this concentration of eugenol, VGSC currents were indeed not affected (FIG. 4C). These results suggest that eugenol reduces excitability of TG neurons and may diminish activity at central terminals to the medullary dorsal horn, the counterpart of spinal dorsal horn in the spinal system [Sessle, B. J., Minerva Anestesiol., 2005, 71: 117-136].

### Experimental Example 3

### Effect of Eugenol on Reversal Potential of Iₕ

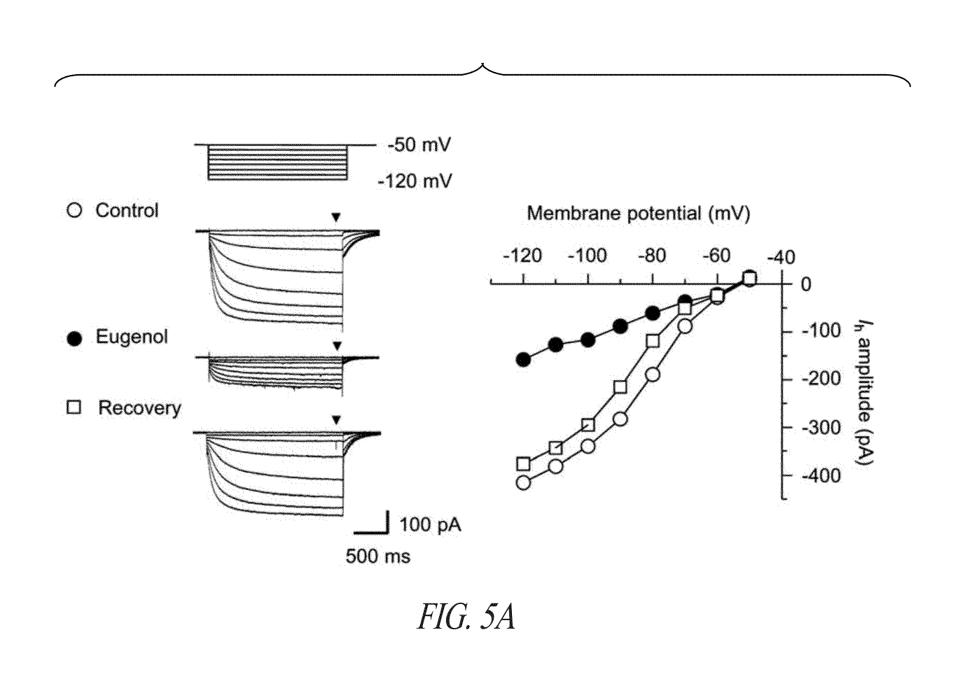

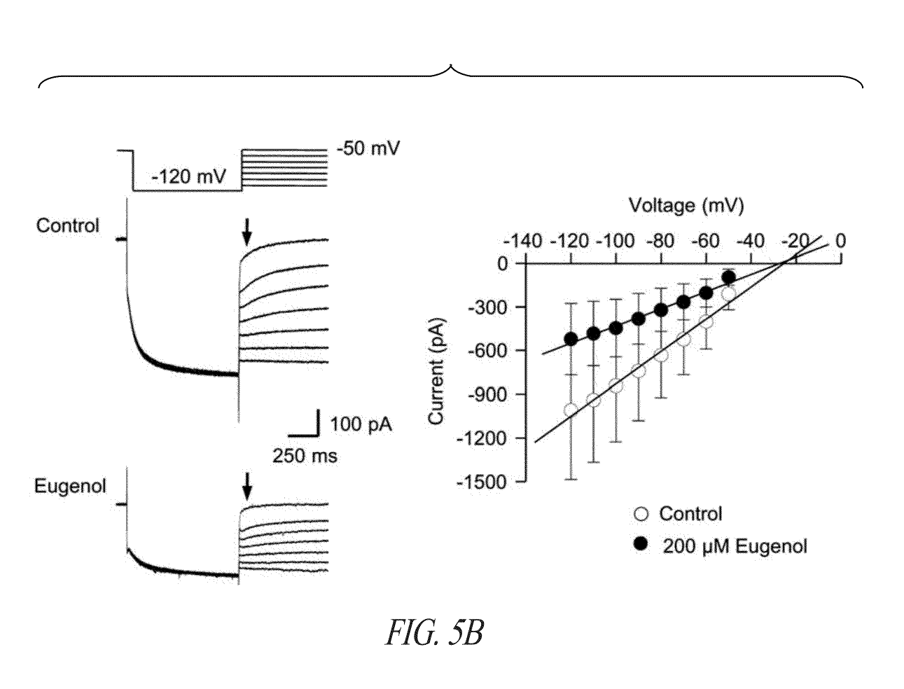

Comparison of steady-state Iₕ currents at the end of a hyperpolarizing step pulses from −50 to −120 mV in increments of 10 mV from a Vₕ of −50 mV indicated that eugenol (200 μM) reduced maximal Iₕ amplitude over the full range of voltages (FIG. 5A, arrows). The reversal potential was measured by first applying a prepulse to −120 mV to fully activate Iₕ and then examining the tail currents after repolarization to test potentials from −110 to −50 mV (FIG. 5B). The time point for measurement of the peak tail current is shown in FIG. 5B (arrows). For each test voltage, the averaged peak tail currents were fitted with a linear regression equation. The mean Vᵣₑᵥ values of −25.8±1.4 mV (n=4) obtained after the application of 200 μM eugenol were not significantly different from the control value, −29.4±2.3 mV (n=4, P>0.05). This reversal potential was consistent with previous reports: −21.3 mV in DRG neurons [Yao, H. et al., J. Neurosci., 2003, 23: 2069-2074], −20.7 mV and −19.1 mV in hHCN1 and hHCN2 channels, respectively [Stieber, J. et al., J. Biol. Chem., 2005, 280: 34635-34643].

### Experimental Example 4

### Mechanism for Inhibitory Action of Eugenol on Iₕ

The mechanism for the inhibitory action of eugenol on Iₕ was investigated. Because Gₛ- or G_{i/o}-protein coupled receptors modulates Iₕ, it was examined whether G_{i/o}-protein participates in the inhibitory effect of eugenol on Iₕ by using eugenol and NEM, a sulpho-alkylating agent which is known to block coupling of GPCR with G_{i/o} [Shapiro, M. S. et al., J. Neurosci., 1994, 14: 7109-7116]. Eugenol-induced Iₕ inhibition was comparable to the Iₕ inhibition after the pretreatment of TG neurons for 5 min with NEM (66.1±1.8% vs 69.2±4.4%; n=8, P>0.05, FIG. 6A), which indicated that the inhibitory effect of eugenol on Iₕ is unlikely to be associated with G_{i/o}-protein coupled receptors.

The cyclic nucleotides, cAMP and cGMP, are known to directly bind and modulate HCN channel gating [Wahl-Schott, C. and Biel, M., Cell Mol. Life. Sci., 2009, 66: 470-494]. Thus, the present inventors tested the involvement of cAMP-mediated signaling in the action of eugenol. They used the cell-permeable cAMP analogue 8-Br-cAMP, and the cAMP isoforms, Sp-cAMP and Rp-cAMP, which are also protein kinase A activators and inhibitors, respectively. 8-Br-cAMP (100 μM) included in the pipette solution significantly attenuated the eugenol-induced Iₕ inhibition (17.5±3.7%; n=6, P<0.001, FIG. 5B). The effect of both Sp-cAMPS and Rp-cAMPs was to also significantly attenuate the eugenol effect (20.1±3.1%, n=5; 25.7±3.4%, n=6; P<0.01; FIG. 6B). These results suggest the inhibitory effect of eugenol is directly related to the cAMP modulation of HCN but not to the downstream activation of PKA by cAMP. Eugenol (200 μM)-induced Iₕ inhibition was inversely proportional to the concentration of 8-Br-cAMP in the pipette solution (FIG. 6C).

### Experimental Example 5

### Effect of Eugenol on Mechanical Allodynia in Trigeminal Neuropathic Pain Model

The effect of eugenol on Iₕ in a trigeminal neuropathic pain model, ION-CCI was further examined. After retrograde DiI labeling, injured TG neurons were identified with a fluorescent microscope (FIG. 7A). The inhibition of Iₕ by 200 μM of eugenol was indistinguishable between the injured TG neurons and the naive TG neurons (67.5±4.0% vs 73.0±3.9%; n=7, P>0.05, FIG. 7A). These results suggest that eugenol could be useful for ameliorating mechanical allodynia in a trigeminal neuropathic pain condition.

Previously, it has been shown that >50 μg eugenol blocks thermal pain in a dose-dependent manner by testing withdrawal reflex in response to noxious thermal stimuli [Park, C. K. et al., Pain, 2009, 144: 84-94]. By measuring air-puff thresholds that induce a head withdrawal reflex, the present inventors found that eugenol significantly ameliorated mechanical allodynia at a lower range of concentrations than those which were effective for ameliorating thermal hyperalgesia (FIG. 7B). As shown in FIG. 7Ba, air-puff thresholds were significantly enhanced with as little as 1 μg eugenol and the threshold reached the cutoff with 5 to 10 μg eugenol. In contrast, consistent with previous finding [Park, C. K. et al., Pain, 2009, 144: 84-94], thermal hyperalgesia was only significantly reduced at 50 μg eugenol, while <10 μg eugenol was not effective (FIG. 7Bb). These results suggest that eugenol at relatively low concentrations could selectively ameliorate mechanical allodynia by inhibiting Iₕ.

In the present invention, it was found that eugenol inhibits VGSCs to reverse mechanical allodynia, at relatively lower concentration than that effectively blocks thermal hyperalgesia, in the trigeminal system, acting via the inhibition of Iₕ currents in TG neurons.

### EFFECT OF THE INVENTION

Even though administered at a concentration lower than that which would inhibit VGSCs, the eugenol of the present invention inhibits A in a cAMP or G-protein coupled receptor independent manner, thereby selectively ameliorating mechanical allodynia. Therefore, when the eugenol of the present invention is formulated into a transdermal preparation to be provided at a concentration lower than that which would inhibit VGSCs, and directly applied to the wound lesion, it can be used as a pharmaceutical composition capable of selectively treating mechanical allodynia.

What is claimed is:

1. A method for treating mechanical allodynia comprising administering to a patient a therapeutically effective amount of eugenol or a pharmaceutically acceptable salt thereof, wherein the concentration of the administered eugenol or a pharmaceutically acceptable salt thereof is lower than that which would inhibit voltage-gated sodium channels (VGSCs).

2. The method according to claim 1, wherein the concentration is lower than that which would inhibit voltage-gated calcium channels (VGCCs).

3. The method according to claim 1, wherein eugenol or the pharmaceutically acceptable salt thereof is administered at a concentration of less than 2 mM.

4. The method according to claim 1, wherein eugenol or the pharmaceutically acceptable salt thereof is administered at a concentration of 1 mM or less.

5. The method according to claim 1, wherein eugenol or the pharmaceutically acceptable salt thereof is administered at a concentration of 200 μM or less.

6. The method according to claim 1, wherein the mechanical allodynia is caused by postsurgical or post-traumatic; metabolic; ischaemic or hemorrhagic; toxic; compression; immune-mediated; inflammatory; or hereditary pain.

7. The method according to claim 1, wherein eugenol or a pharmaceutically acceptable salt thereof is formulated as a transdermal preparation.

8. The method according to claim 7, wherein the preparation is an ointment, a cream, a gel, a lotion, a liquid, an emulsion, a suspension, a stick, a paste, a liniment, a cataplasm, a tape, an aerosol, or an external powder.

9. The method according to claim 7, wherein the preparation includes eugenol or a pharmaceutically acceptable salt thereof in an amount of 0.1 to 2.0% by weight, based on the total weight of the composition.

10. The method according to claim 9, wherein the preparation includes eugenol or a pharmaceutically acceptable salt thereof in an amount of 0.16 to 1.64% by weight, based on the total weight of the composition.

11. The method according to claim 7, wherein the transdermal preparation is applied in an amount of 2 mg/cm² to 20 mg/cm² per unit skin area.

12. A method for blocking hyperpolarization-activated current (Ih) in neurons comprising administering to a patient a therapeutically effective amount of eugenol or a pharmaceutically acceptable salt thereof, wherein the concentration of the administered eugenol or a pharmaceutically acceptable salt thereof is lower than that which would inhibit voltage-gated sodium channels (VGSCs).

13. The method according to claim 12, wherein the concentration is lower than that which would inhibit voltage-gated calcium channels (VGCCs).

14. The method according to claim 12, wherein the eugenol or a pharmaceutically acceptable salt thereof selectively inhibits HCN channels of neurons.
